(19) United States
(12) Patent Application Publication
(10) Pub. No.: US 2025/0285722 A1
(43) Pub. Date: Sep. 11, 2025

(54) SHOCK DETECTION AND MANAGEMENT SYSTEM

(71) Applicant: Nihon Kohden Digital Health Solutions, Inc., Irvine, CA (US)

(72) Inventors: Dexter DE LEON, Tucson, AZ (US); Jessa DECKWA, Casa Grande, AZ (US); Jules BERGMANN, Columbia, MD (US); Harsh DHARWAD, San Diego, CA (US); Mohamed ELMAHDY, Irvine, CA (US); Timothy RUCHTI, Gurnee, IL (US)

(73) Assignee: Nihon Kohden Digital Health Solutions, Inc., Irvine, CA (US)

(21) Appl. No.: 18/598,699

(22) Filed: Mar. 7, 2024

(51) Int. Cl. G16H 20/10; A61B 5/00; A61B 5/02; A61B 5/0215; A61B 5/022; A61B 5/024; A61B 5/08; A61B 5/145; G16H 10/40; G16H 10/60; G16H 20/40

(52) U.S. Cl. CPC G16H 20/10; A61B 5/02028; A61B 5/02152; A61B 5/022; A61B 5/024; A61B 5/0816; A61B 5/14542; A61B 5/14546; A61B 5/6852; A61B 5/743; G16H 10/40; G16H 10/60; G16H 20/40; A61B 2560/0443; A61B 2560/06

(57) ABSTRACT

A clinical care system that integrates and analyzes patient data and applies rules to recommend potential treatments. A potential application is detection and management of shock, using hemodynamic data collected from devices such as a right heart catheter. The system may calculate confidence levels for each rule and present high-ranked treatment options to clinicians along with their confidence levels. Confidence levels for treatments may change continuously as a patient's condition evolves. For shock, features extracted from measured data may include for example a cardiac index (cardiac output divided by patient body surface area), systemic vascular resistance, and mean arterial pressure; treatment recommendations derived from these (and other) features may include administration of various medications such as epinephrine and vasopressin, installation of a ventricular assist device, transfusion, and volume resuscitation. Machine learning may be used to match recommendations to current clinical practice, or to optimize patient outcomes.

### BACKGROUND OF THE INVENTION

### Field of the Invention

One or more embodiments of the invention are related to the field of health care information systems and medical devices. More particularly, but not by way of limitation, one or more embodiments of the invention enable a shock detection and management system.

### Description of the Related Art

Patients in critical condition (or at risk of developing critical complications) must be closely monitored and appropriate treatments must be provided and adjusted quickly to prevent deterioration. In many environments, clinicians must rely on partial information to determine a patient's condition, and they must make care decisions without consulting references or guidelines because of the urgency of the situation. This situation has contributed to relatively high levels of mortality and morbidity in critical care environments.

Patients experiencing or at risk of developing shock present particular challenges for clinicians. Shock is a life-threatening condition that occurs when the body's organs and tissues do not receive an adequate supply of blood. Insufficient perfusion can lead to organ damage, physiological decompensation and, without medical intervention, can be fatal. There are several types of shock, including hypovolemic (due to severe blood or fluid loss), septic (due to infection), anaphylactic (due to allergic reaction), and cardiogenic, among others. Cardiogenic shock occurs when the heart is unable to pump enough blood to the body due to an ischemic heart attack, heart disease, or a condition that directly affects the heart's ability to function, such as cardiomyopathy, heart failure or arrhythmia. When the heart fails to pump blood efficiently, the other organs in the body do not receive the oxygen and nutrients they need to function properly, which can lead to shock. Consequently, shock is an acute condition that requires immediate medical attention to avoid long-term complications and reduce the potential for death.

Often, patients with cardiogenic shock do not receive right-heart catheterization (or pulmonary artery catheterization) which provides critical diagnostic information about the hemodynamic state (pressures and blood flow within the heart) of the patient. Such information is essential to determining the cause and severity of the shock and monitoring the patient's response to treatment.

One reason for the apparent undertreatment is the lack of decision support for associating the complex hemodynamic measurements with actionable information. In addition to the lack of fundamental diagnostic measurements, clinicians treating shock often must manually collect a variety of measurements and perform calculations prior to making decisions. For example, the common measurements used to currently manage shock patients involves the use of information derived from intravenous fluid and drug administration, laboratory measurements, oxygenation and/or mechanical ventilation, vasopressor support, bedside ECG and vital sign monitoring, hemodynamic monitoring, and mechanical circulatory support devices. This information may be used to determine risk scores, such as FICK and PAPI, and for other fundamental calculations, such as pulmonary vascular resistance, through time.

Consequently, clinicians must manage a variety of measurements and data points that vary through time, perform manual averaging and calculations and then, from a wide variety of evidence-based publications, determine the optimal treatment for the patient multiple times per hour.

The urgency of this process is underscored by the patient's acute situation and the deterioration that is accelerated by the body's attempt to compensate for inadequate blood flow. For example, the decreased cardiac output of the diseased heart may activate the sympathetic nervous system leading to the release of adrenaline and noradrenaline which redirect blood to vital organs, increases heart rate and increases the strength of each heart contraction. These mechanisms and others increase the strain on the compromised heart by increasing the resistance against which the heart must pump as well as the amount of blood returning to the heart. The net result of these compensatory actions is a further decrease in cardiac output and an increase in lactic acid build-up (and metabolic acidosis) due to (tissue) hypoxemia.

Consequently, while the clinician is burdened with interpretating the time series of dynamic patient measurements and selecting a supportive intervention, the patient may experience a downward spiral that requires an immediate diagnosis and course of treatment. This challenging situation has likely contributed to the high (27%-51%) in-hospital mortality rate.

Although the medical literature includes various protocols and procedures that describes how to treat the various manifestations of shock as well as how to identify them, there are no known systems that integrate these guidelines into an automated clinical decision support system. There are no known systems that collect and integrate comprehensive data on the condition of a patient at risk for shock, and that analyze this data to provide timely treatment recommendations to clinicians.

For at least the limitations described above there is a need for a shock detection and management system.

### BRIEF SUMMARY OF THE INVENTION

One or more embodiments of the invention may enable a shock detection and management system. The system may provide clinical decision support by collecting and analyzing patient data from multiple sensors or other data sources, and by recommending treatment options based on the analysis.

One or more embodiments of the invention may include a processor that is coupled to a display that is viewable by one or more clinicians that provide care to a patient at risk for shock. The processor may also be coupled to one or more devices that measure or record multiple clinical parameters associated with the patient's physiological status. It may also be coupled to a memory that contains multiple features, each of which is selected from or derived from the clinical parameters. The features may include one or both of cardiac output, and a cardiac index that is the cardiac output divided by the patient's body surface area. The features may also include mean arterial pressure, and systemic vascular resistance that is the cardiac output divided by the mean arterial pressure. The memory may also include several rules, where each rule includes: a treatment recommendation, one or more activation functions that each map a value of a feature into an activation function value, a weight associated with each activation function, and a confidence function that maps feature values into a confidence level that the treatment recommendation is beneficial for the patient. The confidence function may be calculated by applying an aggregation function to the activation function values using the weight associated with each activation function. The processor may be configured to: obtain values of the clinical parameters from the devices, calculate values of the features from the values of the clinical parameters, calculate the confidence level for each rule using the rule's confidence function, select two or more recommended rules that have the highest confidence levels, and transmit the recommended rules and their associated confidence levels to the display. The processor may update the recommended rules and confidence levels over time as values of the clinical parameters change over time.

In one or more embodiments, the patient at risk for shock may be at risk for one or more of cardiogenic shock, hypovolemic shocks, septic shock, and anaphylactic shock.

In one or more embodiments, the features may further include hemoglobin level in blood, mixed venous oxygen saturation, pulmonary capillary wedge pressure, heart rate, and pulmonary vascular resistance.

In one or more embodiments, the treatment recommendations associated with the rules may include: start administration of dobutamine, start administration of milrinone, start administration of clevidipine, start administration of phenylephrine, start administration of norepinephrine, tart administration of vasopressin, and start administration of epinephrine. In one or more embodiments the treatment recommendations may further include: install ventricular assist device, perform transfusion, and perform volume resuscitation.

In one or more embodiments, the devices may include a right heart catheter and an associated hemodynamic monitor. In one or more embodiments, the clinical parameters measured by the right heart catheter may include the cardiac output, the cardiac index, the systemic vascular resistance, pulmonary catheter wedge pressure, central venous pressure, and pulmonary vascular resistance. In one or more embodiments, the devices may further include one or more of a central venous catheter, a sphygmomanometer, a laboratory information system, an electronic medical record, and a noninvasive cardiac output monitor.

In one or more embodiments, the devices may also include a ventricular assist device and a ventilator.

In one or more embodiments, the processor may also be coupled to a user interface via which the clinicians can accept or reject one or more of the recommended rules. In one or more embodiments, the clinicians may also be able to enter notes via the user interface that explain their acceptance or rejection of one or more of the recommended rules. In one or more embodiments, the processor may also be configured to perform an analysis of the notes and the acceptance or rejection of one or more of the recommended rules, and to modify the rules based on this analysis.

In one or more embodiments, the aggregation function may be a weighted average using the weight associated with each activation function and with each rule.

In one or more embodiments, each activation function may be a monotonically nondecreasing or monotonically nonincreasing function of the feature associated with the activation function. In one or more embodiments, each activation function may be a piecewise linear function.

In one or more embodiments, the processor may also be configured to generate one or more plots of the physiological status of the patient based on values of the features, and to transmit these plots to the display. In one or more embodiments, the plots of physiological status may include a two-dimensional plot of the value of the cardiac index on one axis, and the value of the systemic vascular resistance on the other axis.

In one or more embodiments, the system may also include a machine learning system coupled to the processor. The machine learning system may be configured to receive values of the features for multiple patients at risk for shock, receive data on the treatments performed by clinicians on these patients, generate a training dataset with samples having the feature values as inputs and the treatments performed as outputs, train a supervised learning model using the training dataset, and generate the confidence function associated with each rule based on the supervised learning model.

### BRIEF DESCRIPTION OF THE DRAWINGS

The above and other aspects, features and advantages of the invention will be more apparent from the following more particular description thereof, presented in conjunction with the following drawings wherein:

FIG. 1 illustrates a typical process used in the prior art to manage patients at risk for shock: clinicians receive patient information from a number of different sources, and empirically make care decisions based on training and guidelines that may be incomplete or conflicting.

FIG. 2 shows an overview architectural diagram of in embodiment of the invention, which integrates patient data from multiple devices and sources, and applies rules to this data to calculate a set of treatments with the highest degree of confidence for the patient.

FIG. 3 shows an illustrative method of calculating the confidence level for each rule: features are selected from or calculated from measured clinical parameters; the features are input into a set of activation functions associated with each rule, and the activation scores are aggregated into a confidence score for each rule.

FIGS. 4A and 4B contrast a traditional rule guidelines approach based on sharp criteria for when a rule applies (shown in FIG. 4A), with the graduated rule confidence methodology of one or more embodiments of the invention (shown in FIG. 4B).

FIG. 5A shows illustrative devices and data sources that may be used to measure patient clinical parameters, and illustrative features that may be selected or derived from these clinical parameters to drive the rule confidence calculations.

FIG. 5B shows illustrative treatment recommendations that may be associated with a set of rules in one or more embodiments of the invention.

FIG. 6A shows a set of rules in an illustrative embodiment of the invention that uses only three features: cardiac index (CI), systemic vascular resistance (SVR), and mean arterial pressure (MAP).

FIG. 6B shows an expanded set of rules in an illustrative embodiment of the invention that uses 9 features to calculate confidence levels for 12 rules.

FIG. 7 shows illustrative displays of patient state using a two-dimensional grid of the CI and SVR values, and a one-dimensional grid of volume measured by CVP and/or PCWP; these state displays may be shown along with treatment recommendations and their calculated confidence levels.

FIG. 8 illustrates how rules may be cross-referenced to the patient states in which the rules apply.

FIG. 9 illustrates an embodiment of the invention in which clinicians may accept or reject any of the suggested treatments.

FIG. 10 shows an illustrative embodiment of the invention that derives rule confidence functions using machine learning, with a training dataset that may be derived for example from electronic medical records, expert judgements, and clinician treatment selections.

### DETAILED DESCRIPTION OF THE INVENTION

A shock detection and management system will now be described. In the following exemplary description, numerous specific details are set forth in order to provide a more thorough understanding of embodiments of the invention. It will be apparent, however, to an artisan of ordinary skill that the present invention may be practiced without incorporating all aspects of the specific details described herein. In other instances, specific features, quantities, or measurements well known to those of ordinary skill in the art have not been described in detail so as not to obscure the invention. Readers should note that although examples of the invention are set forth herein, the claims, and the full scope of any equivalents, are what define the metes and bounds of the invention.

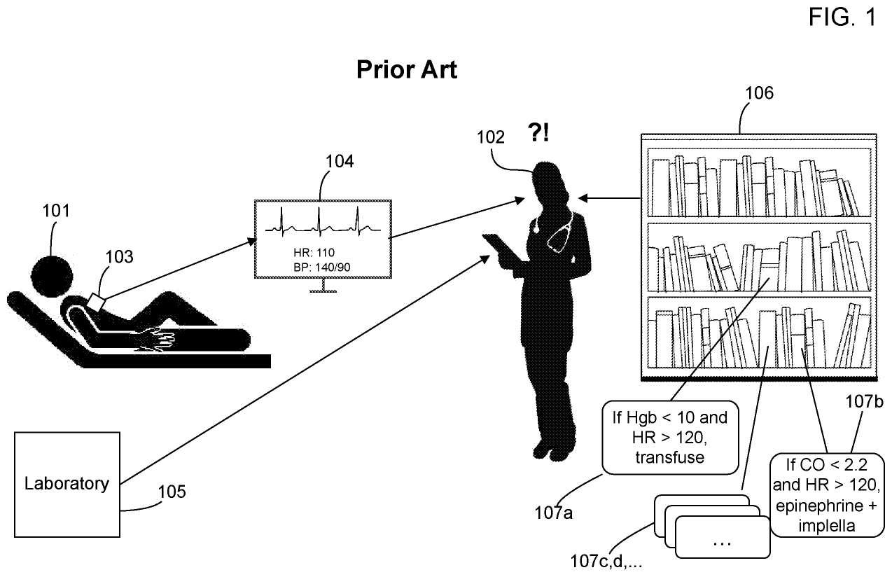

For patients in critical condition (or at risk of developing critical complications), selecting and applying appropriate treatments is both complex and time critical. An important example is patients in cardiac care units who are experiencing or are at risk for shock (cardiogenic, hypovolemic, septic, or anaphylactic for example). Timely and effective treatment is essential to stabilize these patients. However, the optimal treatment and optimal timing depend on many factors. In a typical care setting, many of these factors are either unmeasured or are measured by disparate systems that are not integrated. FIG. 1 illustrates this situation. Patient 101, who may be for example in a cardiac care unit, is at risk for shock. The patient is currently monitored by device 103 that provides information to display 104. Although basic information such as heart rate and blood pressure may be available in real-time, many of the complex hemodynamic factors of the patient's condition are not directly measured. Clinician 102 caring for the patient may also receive, for example, results from laboratory tests 105. Based on this relatively limited information, clinician 102 must make treatment decisions quickly and adjust treatments as the patient's condition evolves. These decisions may be based for example on any of a large number of references 106 that provide recommendations for care of cardiac patients. However, clinician 102 likely has no time to consult the references and must rely on prior training and simple rules of thumb. Moreover, references 106 may provide many different rules such as 107a, 107b, 107c, d, etc., which may be contradictory in some situations. It is not feasible for the clinician to remember or to integrate all of these rules manually into a coherent care plan in real time.

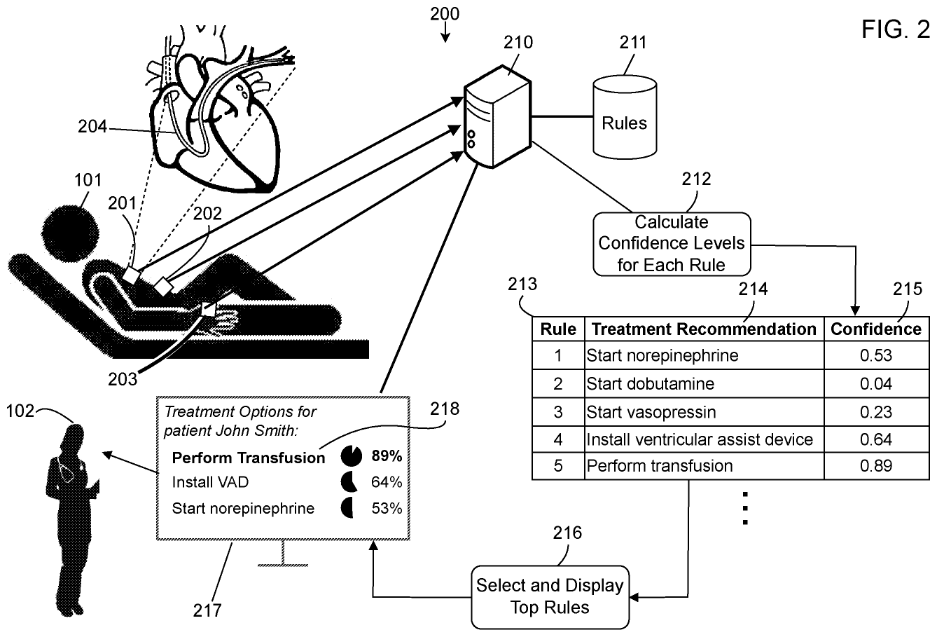

One or more embodiments of the invention may address the issues illustrated in FIG. 1 by integrating patient data from multiple sources and by applying automated rules to the patient data to generate treatment recommendations for the clinician that are updated as the patient condition changes. FIG. 2 shows an overview of an illustrative system 200 with a processor or processors 210 that receive and process data from one or more devices or other data sources that measure the condition of patient 101. Processor(s) 210 may be for example, without limitation, a server, a desktop computer, a laptop computer, a tablet, a smartphone, a CPU, a GPU, or a network of any of these devices. Processor 210 may be coupled to devices and data sources with any type or types of links or network connections. The illustrative embodiment in FIG. 2 shows four devices 201, 202, 203, and 204 coupled to patient 101. These devices may measure any types of clinical or physiological parameters of the patient. Some devices may not be directly coupled to the patient but may provide other data on the patient's status or condition; these devices may include for example, without limitation, an electronic medical record, or a laboratory system. For embodiments that provide clinical decision support for cardiac patients, the devices may include a right heart catheter 204 that is configured to measure hemodynamic parameters such as cardiac output. In one or more embodiments, system 200 may manage data from multiple patients simultaneously, potentially in multiple locations.

Processor 210 may be coupled to a memory that contains a database 211 of rules. Each rule in database 211 has an associated treatment recommendation and information that describes when or to what extent this treatment recommendation is applicable. Database 211 may be organized in any manner (such as SQL or non-SQL databases, files of any format, one or more object stores, or in-memory data structures) and may include code as well as data. Database 211 may include any number of rules. Using rules information 211, processor 210 performs calculations 212 to obtain a confidence level for each of the rules that may apply to patient 101. Output 213 from calculations 212 may include for example a table 213 that describes the treatment recommendation 214 associated with each rule, and the calculated confidence level 215 for the rule given the patient's current state. The confidence level provides a relative measure of the likelihood that the associated treatment would be beneficial or appropriate for patient 101 based on the current or most recently measured parameters obtained from the devices.

After making confidence level calculations 212, processor 210 may then perform step 216 to select a subset of the rules with the highest confidence levels for the current patient state. These top-ranked rules or associated rule descriptions may then be transmitted to a display 217 coupled to the processor and viewable by clinician 102 caring for patient 101. A set of recommendations 218 associated with the top-ranked rules may be displayed along with the calculated confidence levels. A benefit of this system is that multiple recommendations may be displayed along with confidence levels, allowing clinician 102 to select from the top-ranked rules; this approach allows clinicians to apply their own judgement to select an optimal treatment from the options presented, rather than simply presenting a single recommendation with unwarranted certainty.

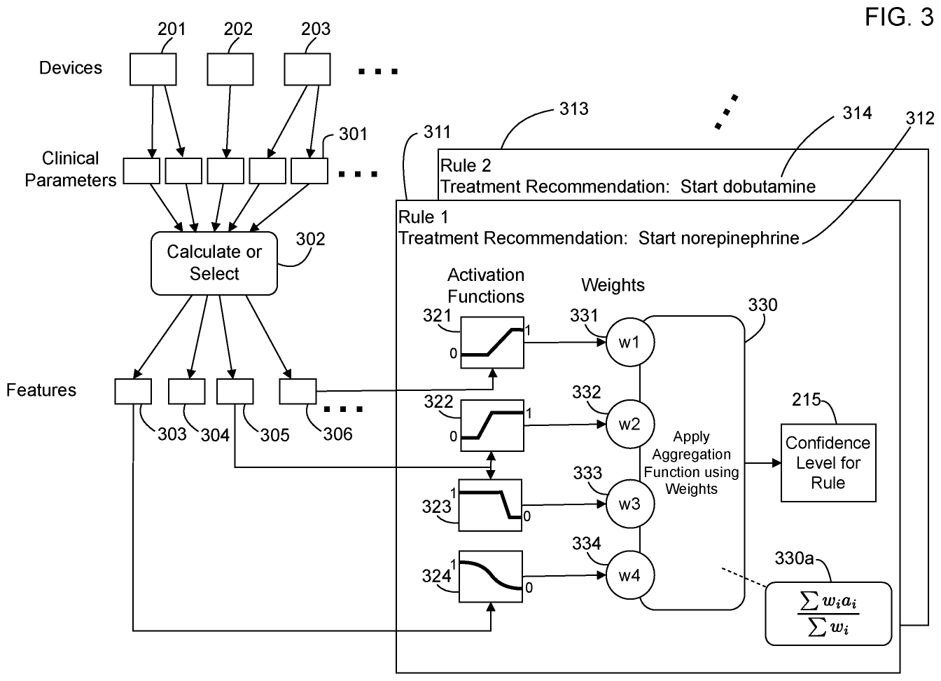

Confidence level calculations 212 may be performed in any manner, using any of the data received from devices and using any algorithms. FIG. 3 illustrates one approach to calculating confidence levels that may be used in one or more embodiments of the invention. In this approach, values of clinical parameters 301 are collected from devices such as 201, 202, 203, etc., and step 302 calculates a set of features such as 303, 304, 305, and 306 from the clinical parameters 301. Calculations 302 may for example perform any data cleaning steps or transformations on the raw clinical parameters 301, such as smoothing, rescaling, filling missing data, and rejection of outliers or implausible values. They may also derive features by combining clinical parameters into features, for example by deriving ratios, sums, linear or nonlinear combinations, minimums, or maximums, etc. Some clinical parameters may be selected directly as features.

Subsequently, separate calculations are performed for each rule to determine the rule's confidence level for the patient's current state. Each rule has an associated treatment recommendation; for example, rule 311 has treatment recommendation 312, and rule 313 has treatment recommendation 314. FIG. 3 illustrates the confidence level calculation for rule 311. Feature values may first be input into activation functions that may reflect whether and to what extent the value of each feature implies that the rule should be activated, and the associated treatment should be recommended. Each rule may have any number of associated activation functions. In this illustrative example, for rule 311 activation functions 321, 322, 323, and 324 each map the value of an associated feature into a value between 0 and 1, where a 0 indicates that the feature has no effect on the rule confidence, and a 1 indicates that the feature has its maximum effect on the rule confidence. The activation functions illustrated are all monotonically nondecreasing (functions 321 and 322) or monotonically nonincreasing (functions 323 and 324). For illustration, activation functions 321, 322, and 323 are piecewise linear, and activation function 324 is a logistic function. Any type of activation function may be used in one or more embodiments of the invention. Some features may be input into more than one activation function; for example, feature 305 is input into activation functions 322 and 323. Some features may not apply to one or more rules, in which case the feature value may not be input into any activation function associated with the rule; for example, feature 304 is not used in rule 311.

The values of activation functions 321, 322, 323, and 324 may then be aggregated in step 330 to calculate the confidence level 215 for the associated rule. Aggregation may use a weight associated with each activation function output, to reflect that activation function's relative importance in determining the rule's confidence level 215. One or more embodiments of the invention may use any types of aggregation functions, and different rules may have different types of aggregation functions. Aggregation functions may include for example, weighted sums or averages, weighted products or geometric means, sums of products or products of sums, or minimums or maximums. For example, aggregation function 330 for rule 311 may be a weighted average 330a that multiplies each activation function output by the associated weight, sums these products, and divides the result by the sum of the weights. This illustrative formula 330a implies that the confidence level 215 will be 1 when all activation functions output 1 and will be 0 when all activation functions output 0.

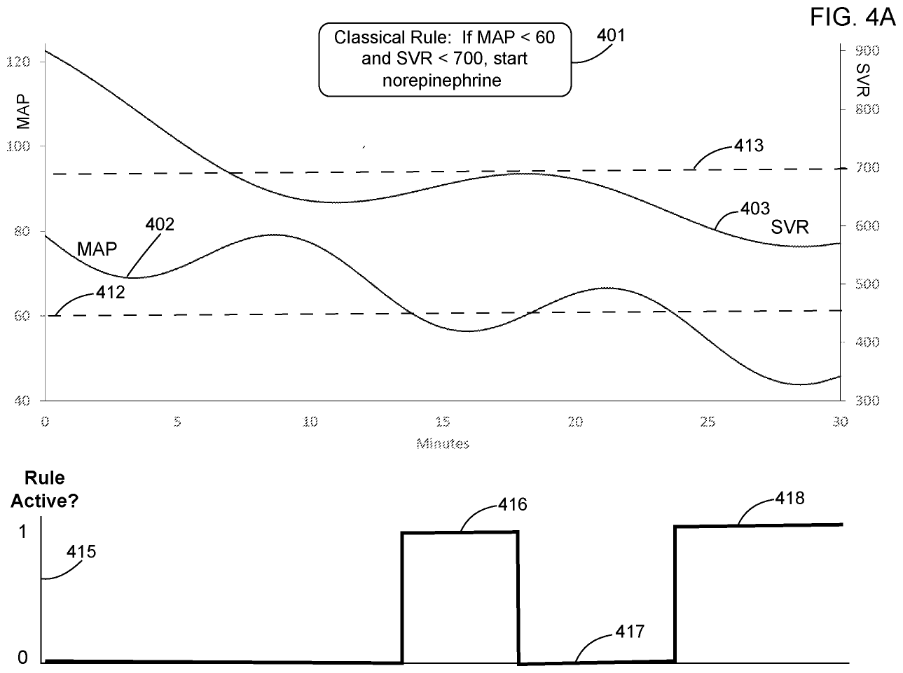

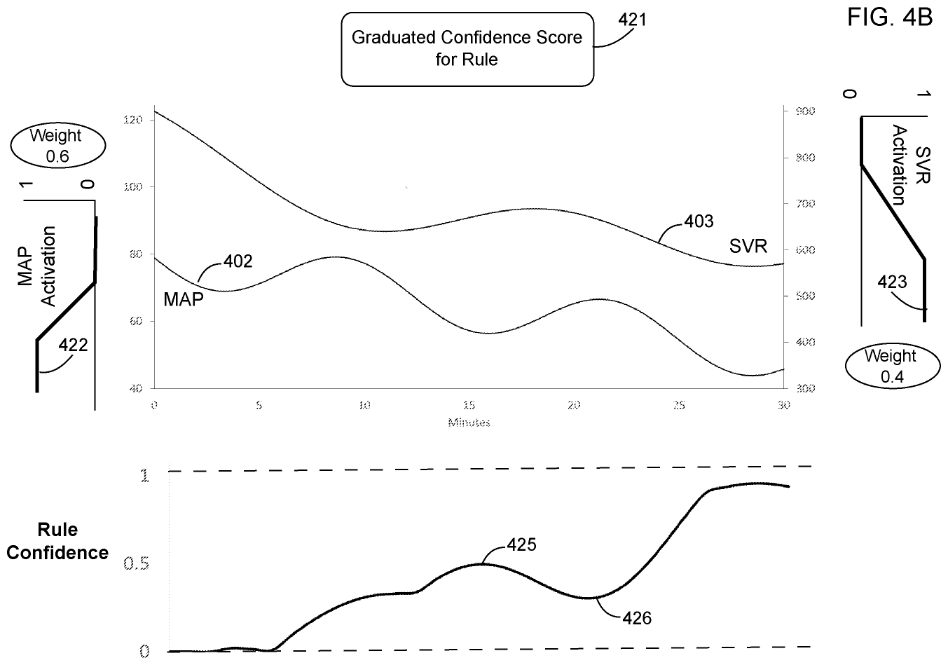

The rule confidence level calculation illustrated in FIG. 3 implies that that the confidence level for a rule will change gradually and continuously as values of the features change over time. This approach is substantially different from traditional clinical guidelines, which typically recommend a specific treatment only when features are within a specific range. FIGS. 4A and 4B illustrate the difference between the classical rule approach (shown in FIG. 4A), and the graduated confidence level approach used in one or more embodiments of the invention (shown in FIG. 4B). In FIG. 4A, a classical rule 401 for treatment of cardiac patients is applied to two features: MAP (mean arterial pressure) and SVR (systemic vascular resistance). This rule is a binary on/off rule with sharp activation boundaries. Graphs 402 of MAP over time (on the left vertical axis) and 403 of SVR over time (or the right vertical axis) show the evolution of an illustrative patient whose condition may be deteriorating. Given classical rule 401, the rule is active when SVR 403 falls below threshold value 413 and MAP 402 falls below threshold value 412. Graph 415 shows the corresponding activation of rule 401, with a 1 value indicating that the rule is active, and 0 indicating that the rule is not active. In this example, rule 401 is briefly activated during period 416, and then becomes inactive during period 417, only to become active again in period 418. This on/off behavior does not reflect the continuous change in the patient's condition and may mislead clinicians more than it assists them.

In contrast, FIG. 4B shows a graduated confidence level approach 421 for rule 401, with the same patient evolution 402 and 403. An activation function 422 is applied to MAP value 402, and activation function 423 is applied to SVR value 403; the activation function outputs are combined in a weighted average using weights 0.6 for MAP and 0.4 for SVR. (These specific activation functions and weights are illustrative.) The resulting rule confidence value 425 changes continuously as the patient condition evolves, increasing gradually from 0 at the beginning of the time interval to a value near 1.0 at the end of the time interval. During period 417 (in FIG. 4A) when the classical rule is deactivated since the MAP value fluctuates briefly above threshold 412, the confidence level for graduated rule 421 dips slightly to value 426, but it does not go immediately to 0. This continuous change in rule confidence reflects the evolving patient condition more directly than the on/off behavior of the classical rule and may be more intuitive for a clinician to evaluate and use.

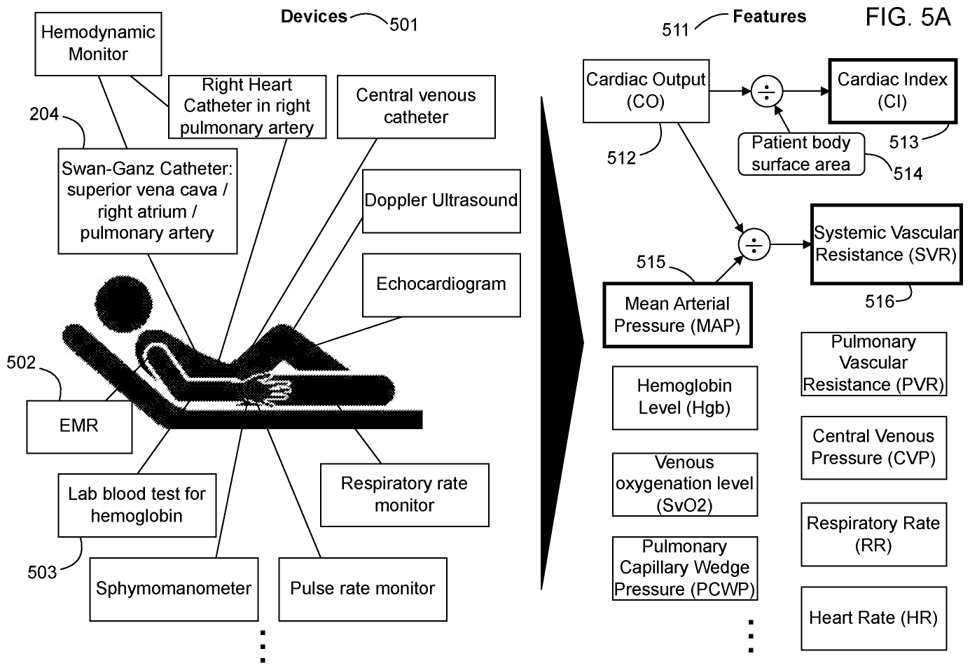

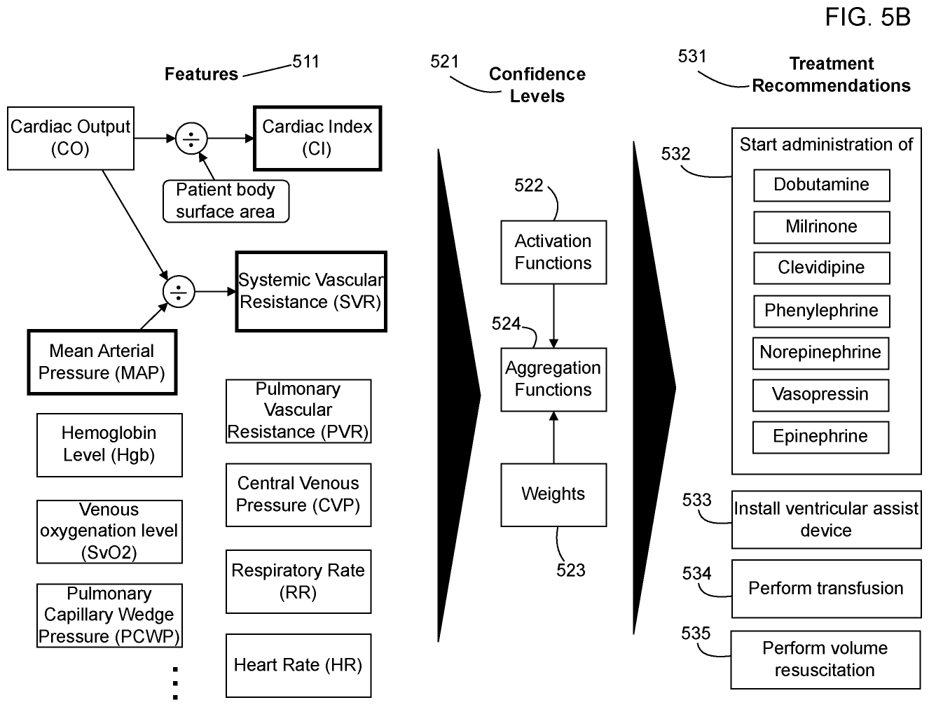

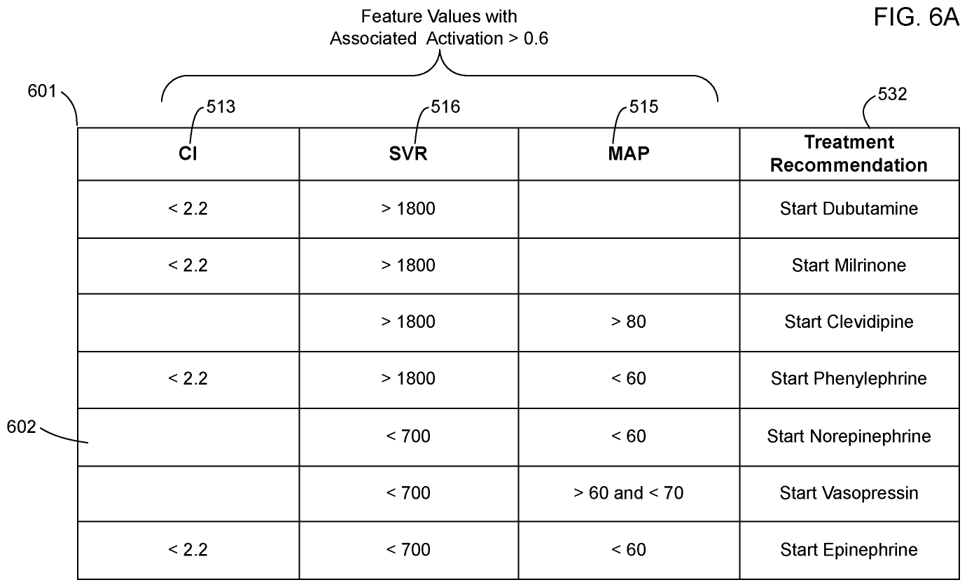

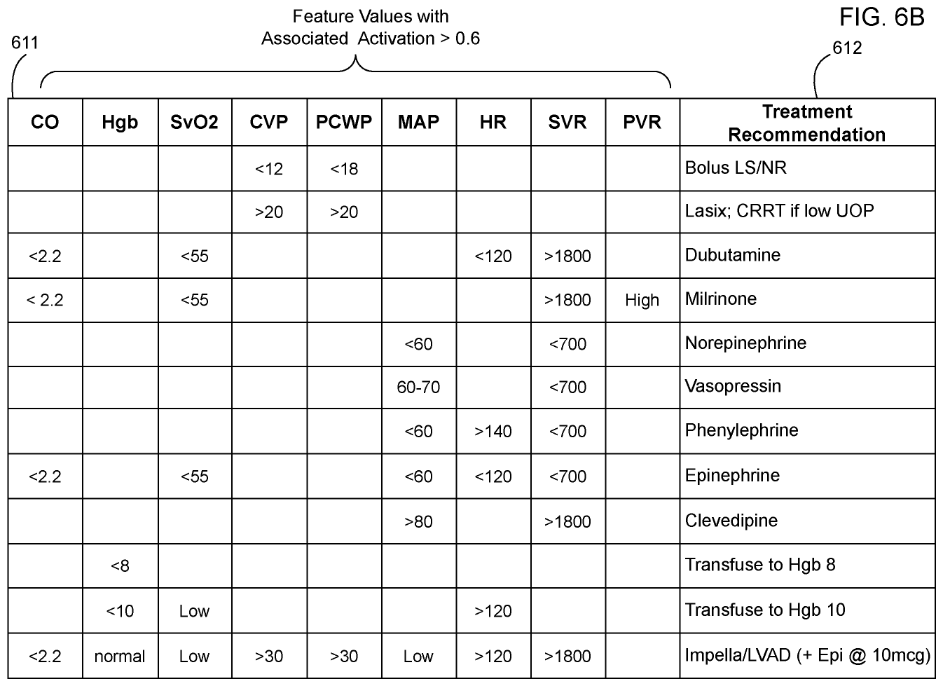

The framework described above for generating confidence levels for a set of rules based on patient data may be applied to any types of patient conditions. FIGS. 5A, 5B, 6A, and 6B describe specific embodiments that may be applied for example to cardiac patients that are at risk for shock. FIG. 5A shows illustrative devices 501 that may be used to collect patient data. These devices may include for example a Swan-Ganz catheter 204 with an associated hemodynamic monitor that collects various parameters of heart function. They may also include systems not directly coupled to the patient, such as an electronic medical record (EMR) system 502, and a laboratory system 503 that may for example analyze the patient's blood for hemoglobin level or other characteristics. Other devices may include for example, without limitation, any type of heart catheter or central venous catheter, a Doppler ultrasound, an echocardiogram, a sphygmomanometer, a pulse rate monitor, and a respiratory rate monitor. One or more embodiments may use any combinations or subsets of these devices, or any additional devices as needed.

FIG. 5A also shows illustrative features 511 that may be selected from or derived from the clinical parameters measured by devices 501. Cardiac Output (CO) 512 may be defined for example as the amount of blood pumped per minute, which may be measured for example by a right heart catheter (or other devices). Cardiac Index (CI) 513 may be defined for example as CO 512 divided by the patient's body surface area 514. Body Surface Area 514 may be estimated for example using the formula: Body Surface Area=(0.007184)*(Height(cm)\hat{ }0.725)*(Weight(kg)\hat{ }0.425). Mean Arterial Pressure (MAP) 515 may be calculated for example as a weighted average of the systolic and diastolic pressure, using for example the formula: MAP=(⅓)*SP+(⅔)*DP. Systemic Vascular Resistance (SVR) 516 may be calculated for example as CO 512 divided by MAP 515. The three features 513, 515, and 516 may be used in one or more embodiments as a minimal set of features to drive calculations of confidence levels for certain rules, as described below with respect to FIG. 6A. Other illustrative features that may be used in one or more embodiments of the invention may include, for example, any or all of Hemoglobin Level (Hgb), Mixed Venous Oxygenation Level (SvO2), Pulmonary Capillary Wedge Pressure (PCWP), Pulmonary Vascular Resistance (PVR), Central Venous Pressure (CVP), Respiratory Rate (RR), and Heart Rate (HR).

The table below provides a summary of features 511:

| Symbol | Description | Measured By | Formula |
|---|---|---|---|
| CO | Cardiac Output: the amount of | Right Heart Catheter, in the | Stroke volume |
| | blood your heart pumps per | right pulmonary artery. | x Heart Rate |
| | minute. | Doppler Ultrasound: Uses | |
| | | an ultrasound machine with | |
| | | a special probe that | |
| | | measures the Doppler shift | |
| | | in the returning ultrasound | |
| | | waves to decipher the blood | |
| | | flow rate and volume, both | |
| | | of which lead to the cardiac | |
| | | index. | |
| | | Echocardiogram: Uses two- | |
| | | dimensional ultrasound | |
| | | paired with Doppler shift | |
| | | measurements to elucidate | |
| | | blood flow rate and volume. | |
| | | Modified carbon dioxide | |
| | | Fick method: Utilizes the | |
| | | Fick principle and measures | |
| | | changes in CO2 elimination | |
| | | and end-tidal CO2 (which is | |
| | | a measure of atrial CO2). | |
| Hgb | Hemoglobin: protein that | Hemoglobin blood test, | |
| | carries oxygen and carbon | number of hemoglobin | |
| | dioxide in blood | present in red blood cells. | |
| SvO2 | Indicates the level of | Swan-Ganz Catheter, central | |
| | oxygenation of mixed venous | venous cannulation of the | |
| | blood returning to the heart | superior vena cava or right | |
| | from the body | atrium | |
| PCWP | Pulmonary Capillary Wedge | Swan-Ganz catheter, central | |
| | Pressure: used to assess left | vein and advancing the | |
| | ventricular filling, represent left | catheter into a branch of the | |
| | atrial pressure, and assess mitral | pulmonary artery | |
| | valve function. | | |
| CI | Cardiac Index: Turns cardiac | | CO/Body |
| | output into a normalized value | | Surface Area |
| | that accounts for the body size | | |
| | of the patient | | |
| CVP | Central Venous Pressure: | Measured by a central | |
| | Measure of pressure in the vena | venous catheter placed | |
| | cava, can be used as an | through either the | |
| | estimation of preload and right | subclavian or internal | |
| | atrial pressure | jugular veins. | |
| MAP | Mean Arterial Pressure: | Invasive arterial catheter | MAP = |
| | Average arterial pressure | and non-invasive | 2/3 * DP + |
| | throughout one cardiac cycle, | intermittent | 1/3 * SP |
| | systole, and diastole. | sphygmomanometer are the | |
| | | standard ways to measure | |
| | | both systolic and diastolic | |
| | | blood pressures. Once these | |
| | | values are known, a MAP | |
| | | value can easily be | |
| | | determined. An | |
| | | oscillometric blood pressure | |
| | | device can also be used to | |
| | | measure MAP. | |
| RR | Respiratory Rate: The number | Different technologies are | |
| | of breaths per minute, is highly | available for measuring. In | |
| | regulated to enable cells to | contact-based measuring | |
| | produce the optimum amount of | techniques, the sensor (i.e., | |
| | energy at any given occasion | the element directly affected | |
| | | by the measurand) must be | |
| | | in contact with the subject's | |
| | | body. | |
| HR | Heart Rate: The number of | Where the pulse is palpated | |
| | beats per minute. The intrinsic | on the radial aspect of the | |
| | rate of the SA node is typically | forearm, just proximal to the | |
| | around 60 to 100 beats per | wrist joint. | |
| | minute (BPM). | | |
| SVR | Systemic Vascular Resistance, | Right Heart Catheterization. | SVR = |
| | or, Total peripheral resistance | SVR may be estimated if | CO/MAP |
| | (TPR), is the amount of force | one can get an accurate | |
| | exerted on circulating blood by | blood pressure reading and | |
| | the vasculature of the body. | the patient's cardiac output, | |
| | | which can be estimated | |
| | | using ultrasound data. The | |
| | | BP can be used to calculate | |
| | | the MAP, and this can be | |
| | | plugged into the above | |
| | | equation to calculate SVR. | |
| PVR | Pulmonary Vascular | This measurement is | |
| | Resistance: resistance against | obtained through a right | |
| | blood flow from the pulmonary | heart catheterization (e.g., | |
| | artery to the left atrium. | Swan-Ganz catheters). | |

FIG. 5B shows illustrative treatment recommendations 531 that may be generated in one or more embodiments of the invention via confidence level calculations 521 based on values of features 511. As described above, in one or more embodiments the confidence level 521 for each treatment recommendation may be calculated using activation functions 522 associated with features 511, whose outputs are combined using weights 523 and an aggregation function 524. Treatment recommendations 532 each recommend administration of various medications, including for example dobutamine, milrinone, clevidipine, phenylephrine, norepinephrine, vasopressin, and epinephrine. In some embodiments a rule may have a treatment recommendation for a specific quantity of an associated medication. Other treatment recommendations associated with rules in one or more embodiments may include for example, without limitation, installation of a ventricular assist device (VAD) 533, performing a transfusion 534, and performing volume resuscitation (535).

FIGS. 6A and 6B show two different sets of rules that may be used in one or more embodiments to manage the risk of shock. FIG. 6A shows a relatively small set of rules 601 that are based on the values of only three features: Cardiac Index 513, Systemic Vascular Resistance 516, and Mean Arterial Pressure 515. Each rule has an associated treatment recommendation 532. Table 601 shows the ranges of each feature within which each rule has an associated activation function that exceeds a threshold value, such as 0.60 for example; features without ranges associated with a rule do not have an associated activation function for that rule. For example, rule 602 may have activation functions 423 for SVR and 422 for MAP as shown in FIG. 4B. FIG. 6B shows a more extensive set of illustrative rules 611 that depend upon the values of 9 different features; these rules also incorporate a wider range of treatment recommendations 612. As in FIG. 6A, the ranges shown for features may correspond to feature values with corresponding activation function values greater than a threshold such as 0.60.

The specific rules, treatment recommendations, features, and activation ranges shown in FIGS. 6A and 6B are illustrative. One or more embodiments of the invention may use different combinations of features, different ranges, different activation functions and weights, and different treatment recommendations.

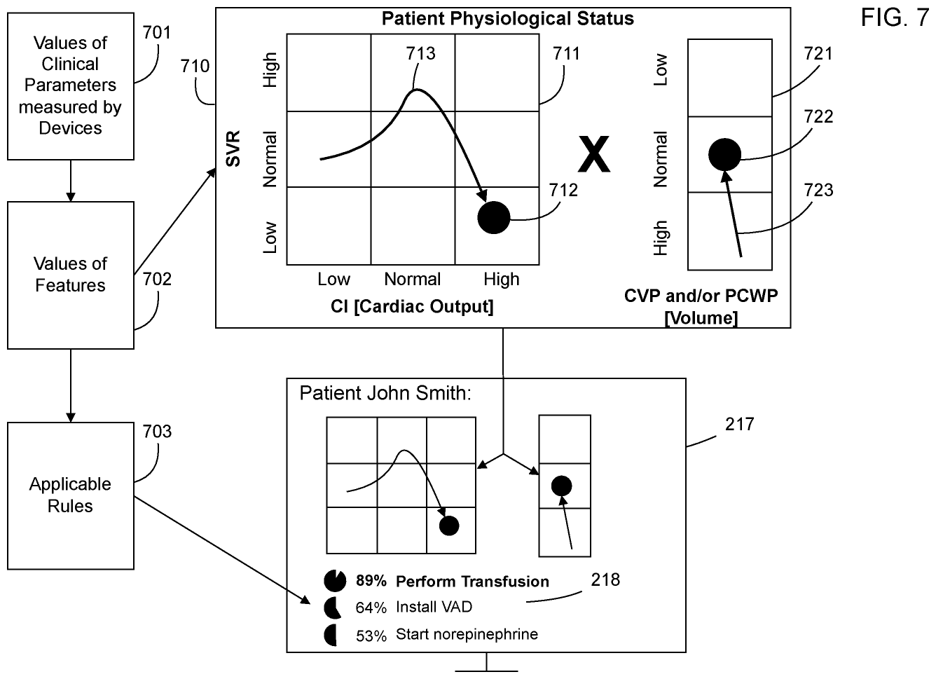

In one or more embodiments of the invention, the system may generate plots or other displays of the patient's physiological status, in addition to calculating rule confidence levels and displaying the top-ranked treatment recommendations. Selected feature values may be plotted for example on one-dimensional or two-dimensional charts or grids. These plots may aid the clinician in understanding the patient's current condition and trajectory, and thereby in understanding why certain treatments are recommended. FIG. 7 shows an illustrative example for patients at risk of shock. As described above, values of clinical parameters 701 are measured by devices, values of features 702 are derived from these clinical parameters, and confidence levels calculated from feature values are used to select a set of applicable rules 703 with treatment recommendations. Feature values 702 may also be used to generate one or more plots of the patient's physiological status. In this example, plot 711 is a two-dimensional display with the Cardiac Index (which measures cardiac output) on one axis, and the Systemic Vascular Resistance on the other axis. Point 712 shows the patient's current state, and trajectory 713 shows the change in the patient's state over time. The patient's volume status, measured by Central Venous Pressure and or Pulmonary Capillary Wedge Pressure, is shown in plot 721; point 722 shows the current value, and trajectory 723 shows the change over time. These plots 711 and 721 may be shown on display 217, along with the recommended treatments 218 and their associated confidence levels.

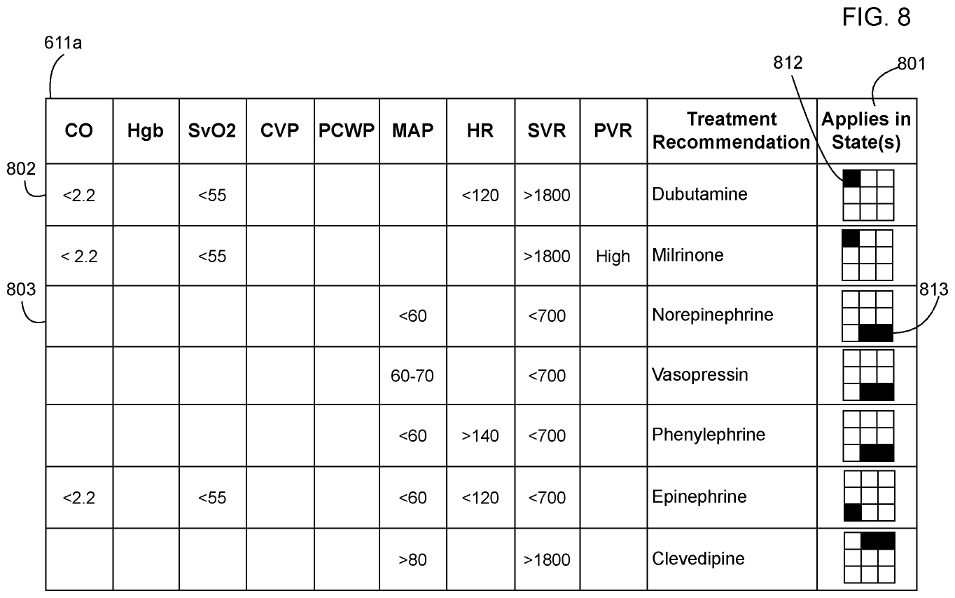

Because the patient status plots and the rule confidence level calculations are both based on the current values of the features, the rule confidence levels are correlated with the patient status plots. The 3-by-3 grid 711 and 1-by-3 grid 721 may therefore provide a useful method for organizing which rules are applicable in which patient states. This methodology may also help clinicians understand why certain rules are recommended with high confidence in certain states. FIG. 8 shows a subset 611a of the rules 611 of FIG. 6B, along with a plot 801 for each rule that shows which portions of the grid 711 each rule is applicable (with a high confidence level). A similar scheme may be used for grid 721, or for any other type of plot of patient status. For example, rule 802 is applicable with high confidence in the upper left grid square 812, where Cardiac Output is low and Systemic Vascular Resistance is high. Similarly rule 802 is applicable in the lower right grid squares 813, where Cardiac Output is moderate or high, and Systemic Vascular Resistance is low.

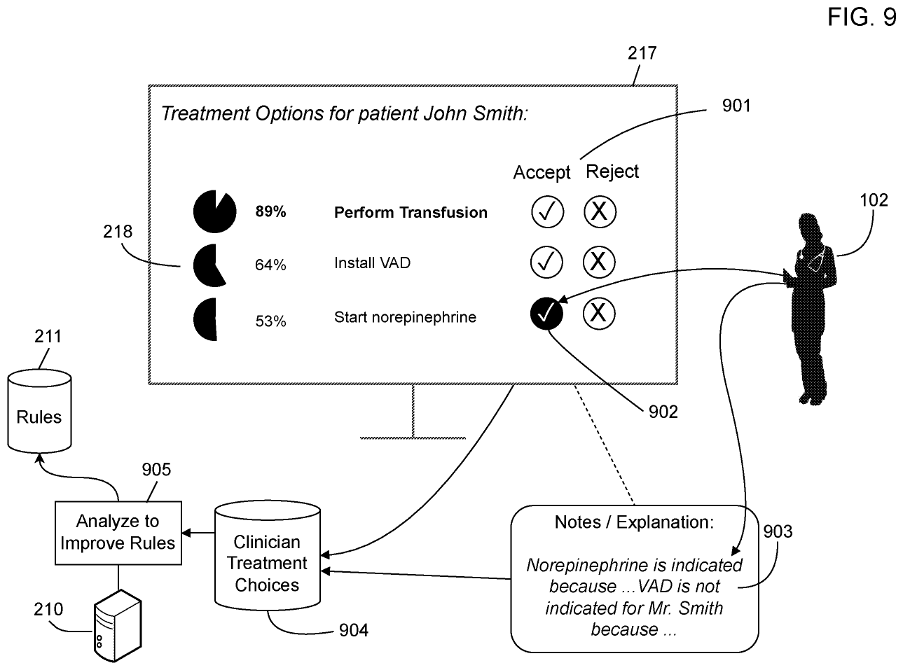

In one or more embodiments of the invention, clinicians may have the capability to indicate whether they accept or reject any or all of the recommended treatments, and potentially to explain their reasoning. This information may be used for example to improve the recommendation system over time based on clinician input. FIG. 9 shows an illustrative embodiment with treatment recommendations 218 shown on display 217; the display also provides selections 901 for clinician 102 to accept or reject each recommendation. In this example the clinician clicks on or otherwise selects button 902 to indicate that the “start norepinephrine” recommendation is accepted. Clinician 102 may also be able to enter notes 903 explaining this decision. A record of the treatments presented 218, the choice made 902, the clinician notes 903, and the patient's state at the time of the choice may be saved in a database 904 of clinician treatment choices; this information may be collected in database 904 for a set of patients over a period of time. Processor 210 (or any other processor or processors) may perform analysis or analyses 905 of database 904, and the results of the analysis may be usedto modify the clinical decision support system and the rules 211, for example by adjusting rule confidence calculations to conform more closely to clinician's actual decisions or to improve patient outcomes.

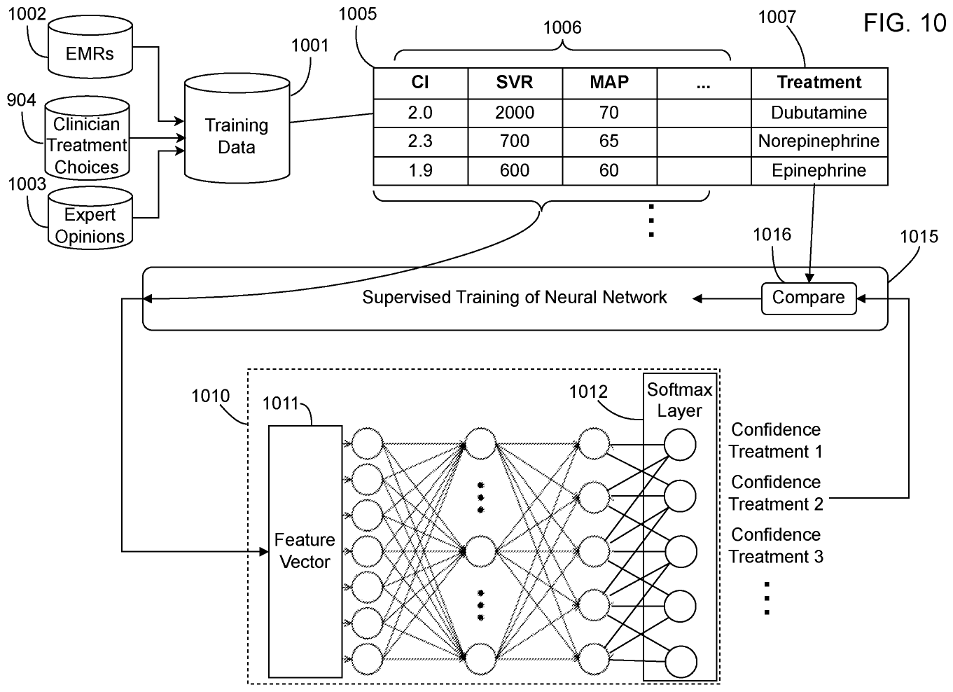

In one or more embodiments, machine learning techniques may be used to train a system to calculate confidence levels for rules, and to improve these calculations over time. FIG. 10 illustrates an approach to machine learning that uses supervised learning to train a system to match clinician's actual or preferred treatment choices. Training data 1001 may for example consist of a collection 1005 of labelled samples, with feature vectors 1006 that may correspond for example to any or all of the features described above, and with the label 1007 indicating the selected or desired treatment. Sources for training data 1001 may include for example, without limitation, electronic medical records 1002, clinician treatment choices 904 captured by the clinical decision support system (as described above with respect to FIG. 9), and expert opinions 1003 on what treatments are best for certain feature vectors. One or more embodiments may use any type of machine learning, including but not limited to neural networks, linear or logistic regression, decision trees, random forests, support vector machines, or nearest neighbors. In the illustrative example shown in FIG. 10, a neural network 1010 is trained in process 1015 using data 1001; feature vector values 1006 are input into the input layer 1011 of the network, and the output layer 1012 may be for example a softmax layer that generates confidence levels for each treatment. The confidence levels calculated by the neural network 1010 may be compared in step 1016 to the actual treatments 1007 (for example with a standard loss function), and the network may be iteratively trained using backpropagation.

Another approach to developing and improving treatment recommendations that may be used in one or more embodiments is to use machine learning techniques to determine treatments that optimize patient outcomes (as opposed to the methodology shown in FIG. 10 that trains the system to match current clinical practice). This approach may require a more extensive training dataset that captures both short-term and long-term patient outcomes, along with the patient status over time and the treatment decisions made. For example, detrimental outcomes may include obvious events such as mortality, as well detrimental exposures (typically exposure to medical interventions such as mechanical ventilation, that increase morbidity and mortality). A cost may be assigned to each type of outcome (for example with higher costs for poorer outcomes), and a system may be trained to perform an optimal action at each point in time to minimize total patient costs.

While the invention herein disclosed has been described by means of specific embodiments and applications thereof, numerous modifications and variations could be made thereto by those skilled in the art without departing from the scope of the invention set forth in the claims.

What is claimed is:

1. A shock detection and management system comprising:
a processor coupled to
a display viewable by one or more clinicians that provide care to a patient at risk for shock;
one or more devices that measure or record a plurality of clinical parameters associated with a physiological status of the patient; and
a memory comprising
a plurality of features, each selected from or derived from the plurality of clinical parameters, wherein the plurality of features comprise
one or both of
cardiac output; and
cardiac index, comprising the cardiac output divided by a body surface area of the patient;
mean arterial pressure; and
systemic vascular resistance, comprising the cardiac output divided by the mean arterial pressure; and,
a multiplicity of rules, wherein each rule comprises
a treatment recommendation;
one or more activation functions associated with each rule, wherein each activation function of the one or more activation functions maps a value of a feature of the plurality of features into an activation function value;
a weight associated with each activation function and with each rule; and
a confidence function associated with each rule that maps values of the plurality of features into a confidence level that the treatment recommendation is beneficial for the patient, wherein the confidence function is calculated by applying an aggregation function to activation function values associated with each rule using the weight associated with each activation function and with each rule;
wherein the processor is configured to
obtain values of the plurality of clinical parameters from the one or more devices;
calculate values of the plurality of features from the values of the plurality of clinical parameters;
calculate the confidence level for each rule of the multiplicity of rules using the confidence function associated with each rule;
select a plurality of recommended rules from the multiplicity of rules that have highest confidence levels;
transmit the plurality of recommended rules and their associated confidence levels to the display; and
update the plurality of recommended rules and associated confidence levels over time as values of the plurality of clinical parameters change over time.

2. The shock detection and management system of claim 1, wherein the patient at risk for shock is at risk for one or more of cardiogenic shock, hypovolemic shock, septic shock, and anaphylactic shock.

3. The shock detection and management system of claim 1, wherein the plurality of features further comprises:
hemoglobin level in blood;
mixed venous oxygen saturation;
pulmonary capillary wedge pressure;
central venous pressure;
heart rate; and,
pulmonary vascular resistance.

4. The shock detection and management system of claim 1, wherein treatment recommendations associated with the multiplicity of rules comprise:
start administration of dobutamine;
start administration of milrinone;
start administration of clevidipine;
start administration of phenylephrine;
start administration of norepinephrine;
start administration of vasopressin; and,
start administration of epinephrine.

5. The shock detection and management system of claim 4, wherein treatment recommendations associated with the multiplicity of rules further comprise:
install ventricular assist device;
perform transfusion; and,
perform volume resuscitation.

6. The shock detection and management system of claim 1, wherein the one or more devices comprise a right heart catheter and an associated hemodynamic monitor.

7. The shock detection and management system of claim 6, wherein clinical parameters measured by the right heart catheter comprise the cardiac output, the cardiac index, the systemic vascular resistance, pulmonary catheter wedge pressure, central venous pressure, and pulmonary vascular resistance.

8. The shock detection and management system of claim 1, wherein the one or more devices further comprise one or more of
a central venous catheter;
a sphygmomanometer;
a laboratory information system;
an electronic medical record system; and,
a noninvasive cardiac output monitor.

9. The shock detection and management system of claim 1, wherein the one or more devices further comprise one or more of
a ventricular assist device; and,
a ventilator.

10. The shock detection and management system of claim 1, wherein
the processor is further coupled to a user interface via which the one or more clinicians can accept or reject one or more of the plurality of recommended rules.

11. The shock detection and management system of claim 10, wherein
the one or more clinicians can further enter notes via the user interface that explain acceptance or rejection of one or more of the plurality of recommended rules.

12. The shock detection and management system of claim 11, wherein
the processor is further configured to
perform an analysis of the notes and the acceptance or rejection of one or more of the plurality of recommended rules; and
modify the multiplicity of rules based on this analysis.

13. The shock detection and management system of claim 1, wherein
the aggregation function comprises a weighted average using the weight associated with each activation function and with each rule.

14. The shock detection and management system of claim 1, wherein
each activation function is a monotonically nondecreasing or a monotonically nonincreasing function of the feature associated with each activation function.

15. The shock detection and management system of claim 14, wherein
each activation function is piecewise linear function.

16. The shock detection and management system of claim 1, wherein the processor is further configured to:
generate one or more plots of the physiological status of the patient based on values of the plurality of features; and,
transmit the one or more plots to the display.

17. The shock detection and management system of claim 16, wherein
the one or more plots of the physiological status of the patient comprise a two-dimensional plot of a value of the cardiac index on one axis and a value of the systemic vascular resistance on a second axis.

18. The shock detection and management system of claim 1, further comprising
a machine learning system coupled to the processor and configured to
receive values of the plurality of features for a multiplicity of patients at risk for shock;
receive data comprising treatments performed by clinicians on the multiplicity of patients;
generate a training dataset comprising samples having the values of the plurality of features as inputs and treatments performed as outputs;
train a supervised learning model using the training dataset; and,
generate the confidence function associated with each rule based on the supervised learning model.
